(19) United States
(12) Patent Application Publication
(10) Pub. No.: US 2012/0071762 A1
(43) Pub. Date: Mar. 22, 2012

(54) ULTRASOUND DIAGNOSTIC APPARATUS

(75) Inventor: Tomoo SATO, Ashigara-kami-gun (JP)

(73) Assignee: FUJIFILM CORPORATION, Tokyo (JP)

(21) Appl. No.: 13/237,295

(22) Filed: Sep. 20, 2011

(30) Foreign Application Priority Data
Sep. 21, 2010 (JP) 2010-210933
Sep. 21, 2010 (JP) 2010-211031

(51) Int. Cl. A61B 8/00

(52) U.S. Cl. 600/459

(57) ABSTRACT

An ultrasound diagnostic apparatus comprises: an ultrasound probe including a transducer array; a plurality of diagnostic apparatus bodies corresponding to a plurality of parts of the transducer array for transmitting ultrasonic waves through corresponding transducers and processing reception signals from the corresponding transducers, respectively; and a synchronizing signal supply unit for supplying a common clock signal and a common trigger signal to the plurality of diagnostic apparatus bodies for causing the plurality of diagnostic apparatus bodies to operate in synchronism.

### BACKGROUND OF THE INVENTION

The present invention relates to an ultrasound diagnostic apparatus and particularly to a plurality of ultrasound diagnostic apparatus bodies operated in parallel whereby ultrasonic wave transmission and reception are performed from a single ultrasound probe.

Conventionally, ultrasound diagnostic apparatus using ultrasound images have been put to use in the medical field. In general, this type of ultrasound diagnostic apparatus comprises an ultrasound probe equipped with a built-in transducer array and an apparatus body connected to the ultrasound probe. The ultrasound probe transmits ultrasonic waves toward a subject, receives the ultrasonic echoes from the subject, and the apparatus body electrically processes the reception signals to generate an ultrasound image.

In recent years, there have been developed ultrasound diagnostic apparatus of portable type that may be transported to a bed side or to a site where emergency medical care is needed. Such ultrasound diagnostic apparatus are required reduction in size to pursue ease of operation and convenience, which necessitates reduction of scale of transmission/reception circuits, necessarily resulting in a reduced image quality. Thus, many of such ultrasound diagnostic apparatus are used in, for example, initial diagnoses and emergency diagnoses.

Obtaining high image quality ultrasound images requires a high-class ultrasound diagnostic apparatus provided with large-scale ultrasound transmission/reception circuits. Even equipment comprising a plurality of portable ultrasound diagnostic apparatus each having only small-scale ultrasound transmission/reception circuits is unable to acquire high image quality ultrasound images without a high-class ultrasound diagnostic apparatus. If high image quality ultrasound images can be obtained by operating a plurality of ultrasound diagnostic apparatus in parallel each equipped with only small-scale ultrasound transmission/reception circuits, such apparatus will be of significantly great use.

JP 2006-519684 A, for example, describes an ultrasound diagnostic system wherein a portable ultrasound unit is mounted on a docking cart to perform data processing. A reception signal produced by the portable ultrasonic unit is supplied to the docking cart and processed using a high data processing capability, whereupon an ultrasound image is displayed with a high resolution on the monitor provided on the docking cart.

The system described in JP 2006-519684 A, with the portable ultrasound unit mounted on the docking cart, is capable of processing the reception signal with a higher processing capability than the processing capability possessed by the portable ultrasound unit. However, even when the ultrasonic unit is mounted on the docking cart, the scale of the ultrasound transmission/reception circuits thereof, i.e., the number of channels, stays unchanged and the level of ultrasound image quality attained with such system is limited.

### SUMMARY OF THE INVENTION

An object of the present invention is to provide an ultrasound diagnostic apparatus in which a plurality of diagnostic apparatus bodies are operated in parallel to obtain a high quality ultrasound image.

An ultrasound diagnostic apparatus according to a first aspect of the present invention comprises:

an ultrasound probe including a transducer array;

a plurality of diagnostic apparatus bodies corresponding to a plurality of parts of the transducer array for transmitting ultrasonic waves through corresponding transducers and processing reception signals from the corresponding transducers, respectively; and

a synchronizing signal supply means for supplying a common clock signal and a common trigger signal to the plurality of diagnostic apparatus bodies for causing the plurality of diagnostic apparatus bodies to operate in synchronism.

An ultrasound diagnostic apparatus according to a second aspect of the present invention comprises:

an ultrasound probe including a transducer array; and

a plurality of diagnostic apparatus bodies corresponding to a plurality of parts of the transducer array for transmitting ultrasonic waves through corresponding transducers and processing reception signals from the corresponding transducers, respectively,

wherein when the one ultrasound probe is connected to the plurality of diagnostic apparatus bodies, one diagnostic apparatus body is selected as master apparatus body from among the plurality of diagnostic apparatus bodies while the other diagnostic apparatus bodies become slave apparatus bodies for the master apparatus body, and the plurality of diagnostic apparatus bodies operate in synchronism.

### BRIEF DESCRIPTION OF THE DRAWINGS

FIG. 1 is a block diagram illustrating a configuration of an ultrasound diagnostic apparatus according to Embodiment 1 of the invention.

FIG. 2 is a block diagram illustrating a specific configuration of the ultrasound diagnostic apparatus according to Embodiment 1.

FIG. 3 is a flowchart illustrating a flow of operation mode change in diagnostic apparatus units in Embodiment 1.

FIG. 4 is a view illustrating a relationship between diagnostic apparatus units and a transducer array in Embodiment 1.

FIG. 5 illustrates transmission of ultrasound from the transducer array in Embodiment 1.

FIG. 6 illustrates reception of ultrasonic echoes by the transducer array in Embodiment 1.

FIGS. 7A and 7B respectively illustrate beam forming in a first diagnostic apparatus unit and a second diagnostic apparatus unit used in Embodiment 1.

FIG. 8 illustrates a sound ray signal synthesized in Embodiment 1.

FIG. 9 illustrates ultrasonic beam transmitted from the transducer array of an ultrasound probe.

FIGS. 10A to 100 respectively illustrate profiles of ultrasonic beams at frequencies 2 GHz, 40 MHz, and 20 MHz, transmitted from the transducer array of the ultrasound probe.

FIG. 11 is a block diagram illustrating a configuration of an ultrasound diagnostic apparatus according to Embodiment 2.

FIG. 12 is a block diagram illustrating a specific configuration of the ultrasound diagnostic apparatus according to Embodiment 2.

FIG. 13 is a block diagram illustrating an internal configuration of a diagnostic apparatus sub-unit used in Embodiment 2.

FIG. 14 is a timing chart illustrating a relationship between a clock signal and a trigger signal in Embodiment 2.

FIG. 15 is a block diagram illustrating a specific configuration of an ultrasound diagnostic apparatus according to Embodiment 3.

FIG. 16 is a block diagram illustrating a specific configuration of the ultrasound diagnostic apparatus according to a modification of Embodiment 3.

### DETAILED DESCRIPTION OF THE INVENTION

Embodiments of the present invention will be described below based on the appended drawings.

### Embodiment 1

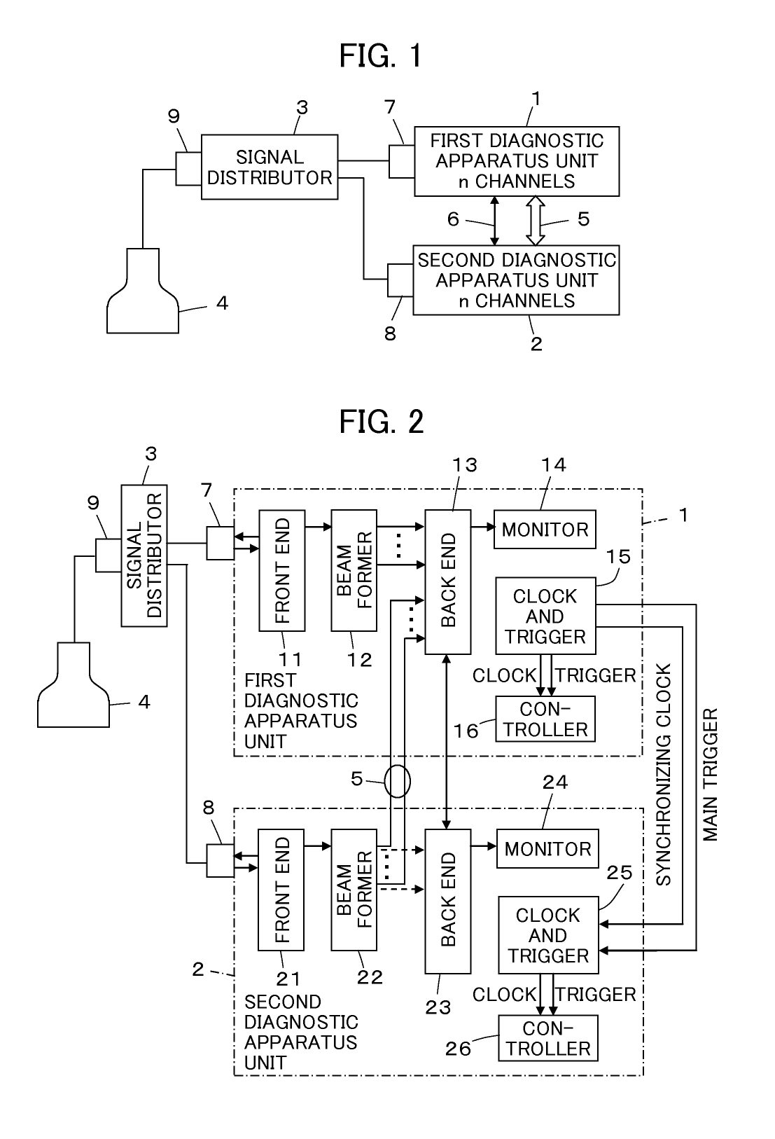

FIG. 1 illustrates a configuration of an ultrasound diagnostic apparatus according to Embodiment 1 of the invention. The ultrasound diagnostic apparatus comprises a first diagnostic apparatus unit 1 and a second diagnostic apparatus unit 2 as two diagnostic apparatus bodies. These first diagnostic apparatus unit 1 and the second diagnostic apparatus unit 2 are connected via a signal distributor 3 to a common ultrasound probe 4.

The first diagnostic apparatus unit 1 and the second diagnostic apparatus unit 2 have an identical inner configuration to each other, each comprising n number of channels of ultrasound transmission/reception circuits, and are connected to each other via a data bus 5 and an operation control cable 6.

The ultrasound probe 4 comprises a transducer array having a number of apertures that is equal to or greater than 2 n, which is the sum of the numbers of channels of the diagnostic apparatus units 1 and 2.

The signal distributor 3 is connected to the first diagnostic apparatus unit 1 and the second diagnostic apparatus unit 2 via unit side connectors 7 and 8 and connected to the ultrasound probe 4 via a probe connector 9. The signal distributor 3 selectively connects some of the transducers constituting the transducer array of the ultrasound probe 4 to the first diagnostic apparatus unit 1 and selectively connects some other transducers, other than those connected to the first diagnostic apparatus unit 1, to the second diagnostic apparatus unit 2.

FIG. 2 illustrates the internal configurations of the first diagnostic apparatus unit 1 and the second diagnostic apparatus unit 2. The first diagnostic apparatus unit 1 comprises a front end 11 connected to the signal distributor 3 via the unit side connector 7. The front end 11 is connected via a beam former 12 to a back end 13, which is connected to a monitor 14. The first diagnostic apparatus unit 1 further comprises a clock retrigger circuit 15, which is connected to a controller 16.

Equipped with transmission and reception circuits having an n number of channels, the front end 11 supplies actuation signals to the corresponding transducers of the ultrasound probe 4, to which the front end 11 is connected via the signal distributor 3, and receives ultrasonic echoes returning from a subject to perform quadrature detection or other processing on reception signals generated by these transducers to produce a complex baseband signal, whereupon the front end 11 performs sampling on the complex baseband signal to produce sample data containing information on an area of a tissue. The front end 11 may produce sampling data by performing data compression processing for high efficiency encoding on the data obtained by sampling the complex baseband signal.

The beam former 12 selects one reception delay pattern from a plurality of previously stored reception delay patterns according to the reception direction set by the controller 16, and based on a selected reception delay pattern, performs the reception focusing processing by providing respective delays in the plurality of complex baseband signals represented by the sample data, and adding them up. By this reception focusing processing, a baseband signal (sound ray signal) in which the focal points of the ultrasonic echoes are made to converge is generated.

The back end 13 produces a B mode image signal, which is tomographic image information on the tissue of the subject, according to the sound ray signal generated by the beam former 12. The back end 13 comprises an STC (sensitivity time control) and a DSC (digital scan converter). For the sound ray signals, the STC corrects attenuation due to distance in accordance with the depth of the reflection location of the ultrasound wave. The DSC performs raster conversion of the sound ray signal corrected by the STC into an image signal compatible with the scanning method of an ordinary television signal, and then, by performing the required image processing such as contrast processing, it generates a B mode image signal.

The monitor 14 displays an ultrasound diagnostic image based on an image signal produced by the back end 13.

The clock retrigger circuit 15 supplies a clock signal to components provided in the diagnostic apparatus unit 1 and supplies a trigger signal retriggered by that clock signal to components provided in the diagnostic apparatus unit 1.

The controller 16 controls operations of components provided inside the diagnostic apparatus unit 1.

The second diagnostic apparatus unit 2 also has like internal configuration as the first diagnostic apparatus unit 1. The second diagnostic apparatus unit 2 comprises a front end 21 connected to the signal distributor 3 via the unit side connector 8. The front end 21 is connected via a beam former 22 to a back end 23, which in turn is connected to a monitor 24. The second diagnostic apparatus unit 2 further comprises a clock retrigger circuit 25, which is connected to a controller 26.

These components provided in the second diagnostic apparatus unit 2 have like functions as those given the same names provided in the first diagnostic apparatus unit 1.

When the first diagnostic apparatus unit 1 and the second diagnostic apparatus unit 2 are in parallel operation, the first diagnostic apparatus unit 1, for example, is selected as master apparatus body to function as such, and the second diagnostic apparatus unit 2 is then selected as slave apparatus body to function as such. In this case, as illustrated in FIG. 2, the beam former 22 of the second diagnostic apparatus unit 2 is connected to the back end 13 of the first diagnostic apparatus unit 1 via the data bus 5, while the back end 23 and the clock retrigger circuit 25 of the second diagnostic apparatus unit 2 are connected via the operation control cable 6 to the back end 13 and the clock retrigger circuit 15 of the first diagnostic apparatus unit 1.

The unit side connectors 7 and 8 connected to the signal distributor 3 are previously assigned different identification numbers (ID numbers), so that the first diagnostic apparatus unit 1 or the second diagnostic apparatus unit 2 recognizes that it is to function as master apparatus body upon connection to the unit side connector 7 by recognizing the ID number assigned to the unit side connector 7 and recognizes that it is to function as slave apparatus body upon connection to the unit side connector 8 by recognizing the ID number assigned to the unit side connector 8.

The probe connector 9 connected to the ultrasound probe 4 is also previously assigned an ID number that is different from those assigned to the unit side connectors 7 and 8 such that when directly connected to the probe connector 9, the first diagnostic apparatus unit 1 and the second diagnostic apparatus unit 2 recognize that they are not to perform parallel operation but independently perform normal ultrasound diagnostic operation.

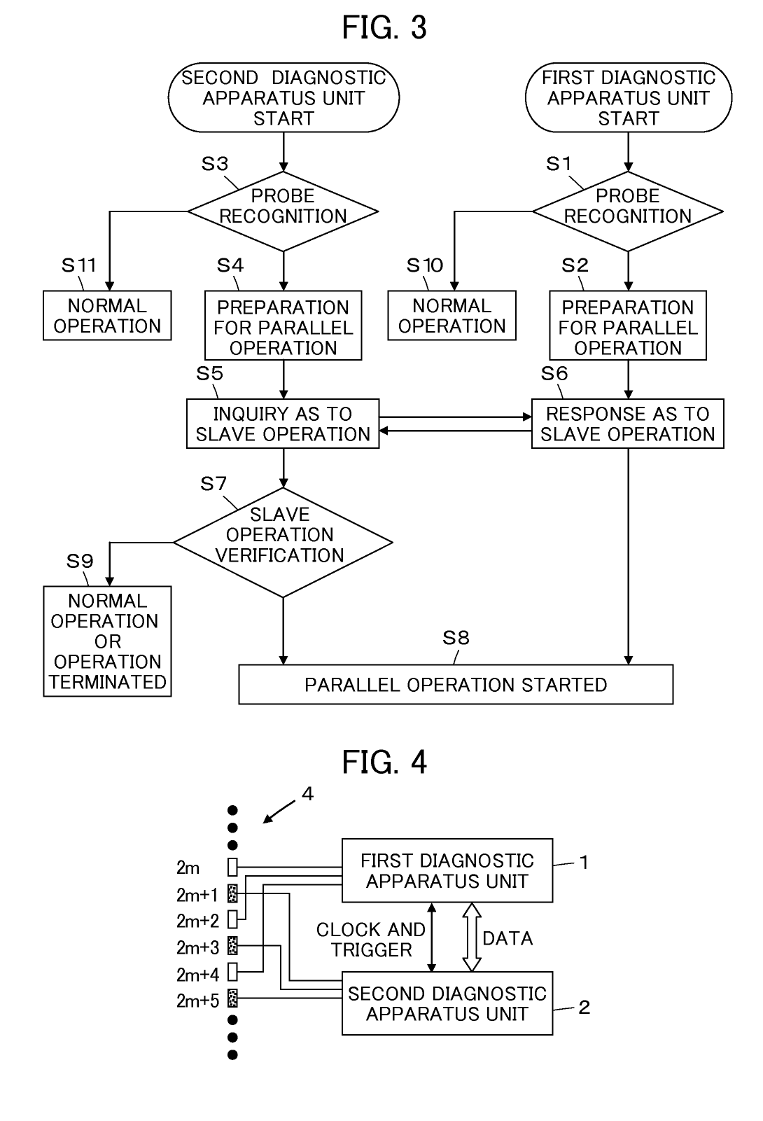

Now, referring to the flowchart illustrated in FIG. 3, a flow of operation mode change in the first diagnostic apparatus unit 1 and the second diagnostic apparatus unit 2 will be described.

First, in step S1, the first diagnostic apparatus unit 1 recognizes whether the ultrasound probe has been directly connected based on the ID number of the coupled connector. As shown in FIG. 2, when connected to the unit side connector 7, the first diagnostic apparatus unit 1 recognizes that it has been selected as master apparatus body and is to perform the parallel operation, proceeding to step S2 to prepare for parallel operation. Specifically, the clock retrigger circuit 15 supplies its own clock signal and trigger signal via the operation control cable 6 to the clock retrigger circuit 25 of the second diagnostic apparatus unit 2 as synchronizing clock signal and main trigger signal, respectively.

In parallel thereto, the second diagnostic apparatus unit 2 recognizes in step S3 whether the ultrasound probe has been directly connected based on the ID number of the coupled connector. As shown in FIG. 2, when connected to the unit side connector 8, the second diagnostic apparatus unit 2 recognizes that it has been selected as salve apparatus body and is to perform the parallel operation, the procedure proceeding to step S4 to prepare for parallel operation. That is, the clock retrigger circuit 25 supplies the synchronizing clock signal and the main trigger signal supplied via the operation control cable 6 from the clock retrigger circuit 15 of the first diagnostic apparatus unit 1 to components provided in the second diagnostic apparatus unit 2.

Then, in step S5, the second diagnostic apparatus unit 2 inquires of the first diagnostic apparatus unit 1 via the operation control cable 6 as to the slave operation, and, when the first diagnostic apparatus unit 1 gives a response as to the slave operation in step S6, verifies the slave operation in step S7. Upon verification that the slave operation is possible, the procedure proceeds to step S8 to start the parallel operation.

On the other hand, the first diagnostic apparatus unit 1, after replying to the second diagnostic apparatus unit 2 as to the slave operation in step S6, proceeds to step S8 to start the parallel operation.

When verification that the slave operation is possible cannot be made in step S7, the procedure proceeds to step S9, where the second diagnostic apparatus unit 2 alone performs a normal ultrasound diagnostic operation or terminates operation.

Upon recognition through the ID number of the coupled connector in step S1 and step S3 that the probe connector 9 has been connected, the first diagnostic apparatus unit 1 and the second diagnostic apparatus unit 2 proceed to step S10 and step S11, respectively, to perform a normal ultrasound diagnostic operation independently.

Next, the parallel operation will be described.

First, as illustrated in FIG. 4, the signal distributor 3 ensures that the first diagnostic apparatus unit 1 is connected to the transducers located in even-number positions in the transducer array of the ultrasound probe 4, and the second diagnostic apparatus unit 2 is connected to the transducers located in odd-number positions.

The second diagnostic apparatus unit 2 to function as slave apparatus body operates according to the synchronizing clock signal and the main trigger signal supplied from the clock retrigger circuit 15 of the first diagnostic apparatus unit 1.

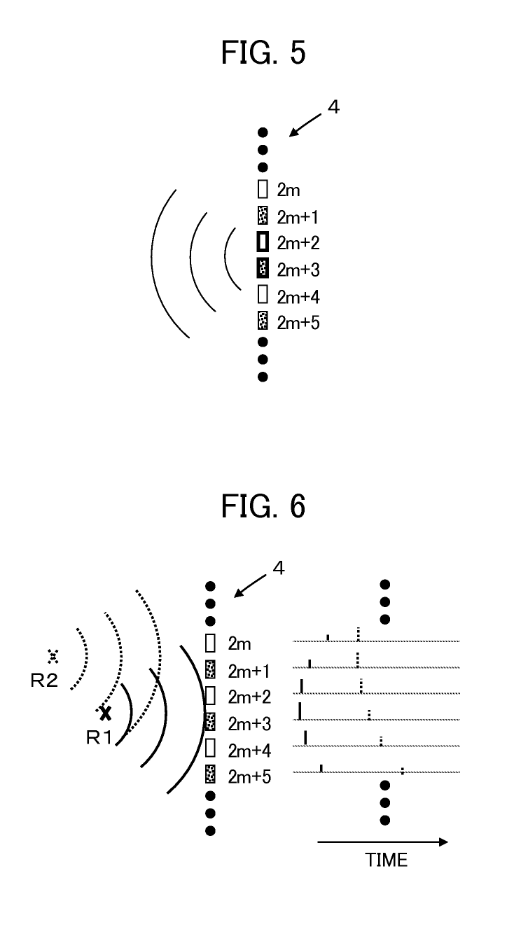

When, for example, the front end 11 of the first diagnostic apparatus unit 1 supples the actuation signal to the (2 m+2)th transducer of the ultrasound probe 4, and when the front end 21 of the second diagnostic apparatus unit 2 supplies the actuation signal to the (2 m +3)th transducer of the ultrasound probe 4, m being a natural number, then, upon transmission of ultrasonic waves from these two transducers located adjacent to each other as illustrated in FIG. 5, the transducers of the transducer array in the ultrasound probe 4 having received ultrasonic echoes from the subject respectively output reception signals as illustrated in FIG. 6.

FIG. 6 shows that two regions of interest R1 and R2 in the subject generate ultrasonic echoes: the reception signal corresponding to the ultrasonic echo from the region of interest R1 is schematically indicated by a solid line; the reception signal corresponding to the ultrasonic echo from the region of interest R2 is schematically indicated by a dotted line;

The reception signal outputted from the transducer located in an even number position in the transducer array is inputted to the front end 11 of the first diagnostic apparatus unit 1 to produce sample data, while the reception signal outputted from the transducer located in an odd number position in the transducer array is inputted to the front end 21 of the second diagnostic apparatus unit 2 to produce sample data. At this time, since the second diagnostic apparatus unit 2 operates according to the synchronizing clock signal and the main trigger signal supplied from the clock retrigger circuit 15 of the first diagnostic apparatus unit 1, the front end 11 of the first diagnostic apparatus unit 1 and the front end 21 of the second diagnostic apparatus unit 2 produce sample data at the same timing as each other.

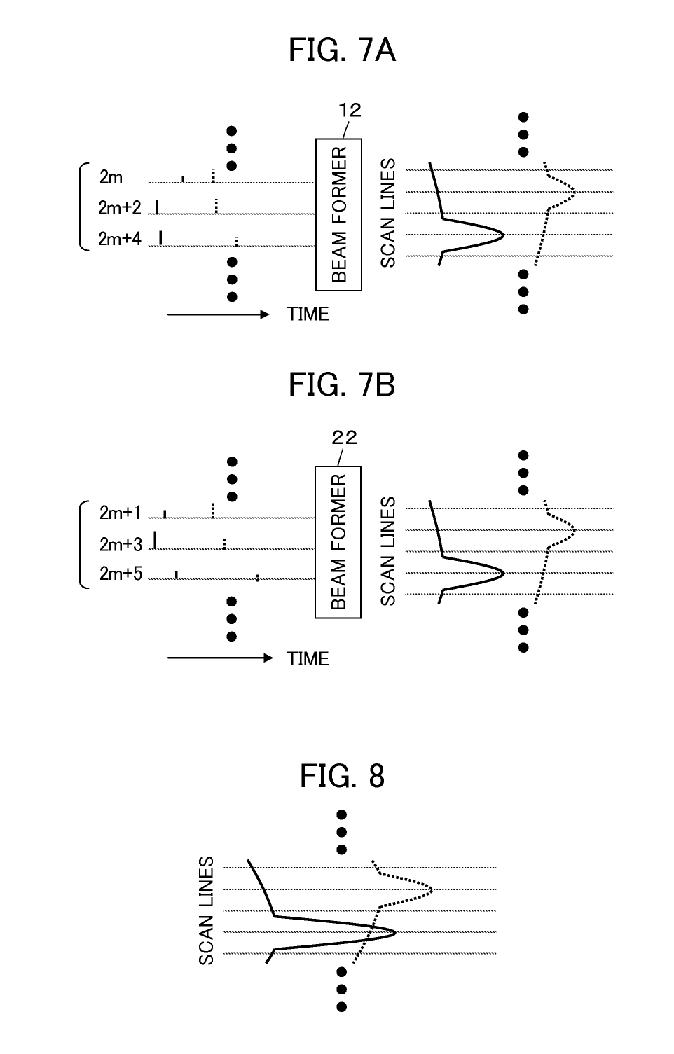

In the first diagnostic apparatus unit 1, as the beam former 12 performs reception focusing processing on the sample data produced by the front end 11, a sound ray signal is produced and supplied to the back end 13 as illustrated in FIG. 7A. Also in the second diagnostic apparatus unit 2, as the beam former 22 performs reception focusing processing on the sample data produced by the front end 21, a sound ray signal is produced as illustrated in FIG. 7B and supplied to the back end 13 of the first diagnostic apparatus unit 1 via the data bus 5.

Here, the first diagnostic apparatus unit 1 and the second diagnostic apparatus unit 2 may be so configured as to make phase adjustment for the transducers each forming respective openings in the transducer array of the ultrasound probe 4 using sub-openings, combines ultrasonic beams traveling in a plurality of directions, and generates a sound ray signal based on the synthesis results.

When supplied with the sound ray signals produced respectively by the beam formers 12 and 22 of both diagnostic apparatus units 1 and 2, the back end 13 of the first diagnostic apparatus unit 1 combines these sound ray signals as illustrated in FIG. 8 and, based on the synthesized sound ray signal, produces the B-mode image signal, which is tomographic image information on the tissue of the subject. This image signal is transmitted to the monitor 14 of the first diagnostic apparatus unit 1, and an ultrasound diagnostic image is displayed on the monitor 14.

Thus, according to Embodiment 1, when the first diagnostic apparatus unit 1 and the second diagnostic apparatus unit 2 are connected to a single ultrasound probe 4 via the signal distributor 3, the first diagnostic apparatus unit 1 functions as master apparatus body, while the second diagnostic apparatus unit 2 functions as slave apparatus body according to the ID numbers of the coupled unit side connectors, and the first diagnostic apparatus unit 1, master apparatus body, supplies the synchronizing clock signal and the main trigger signal to the second diagnostic apparatus unit 2, so that these diagnostic apparatus units 1 and 2 perform the parallel operation.

Since the first diagnostic apparatus unit 1 and the second diagnostic apparatus unit 2 each have an n number of channels of ultrasound transmission/reception circuits, the number of reception signals that can be processed in parallel simultaneously when these units perform a normal ultrasound diagnostic operation independently is “n”. However, when they perform the parallel operation, the number of reception signals that can be processed in parallel simultaneously is “2 n” which is double the number that is possible in independent operation. This enables a high quality ultrasound image to be obtained.

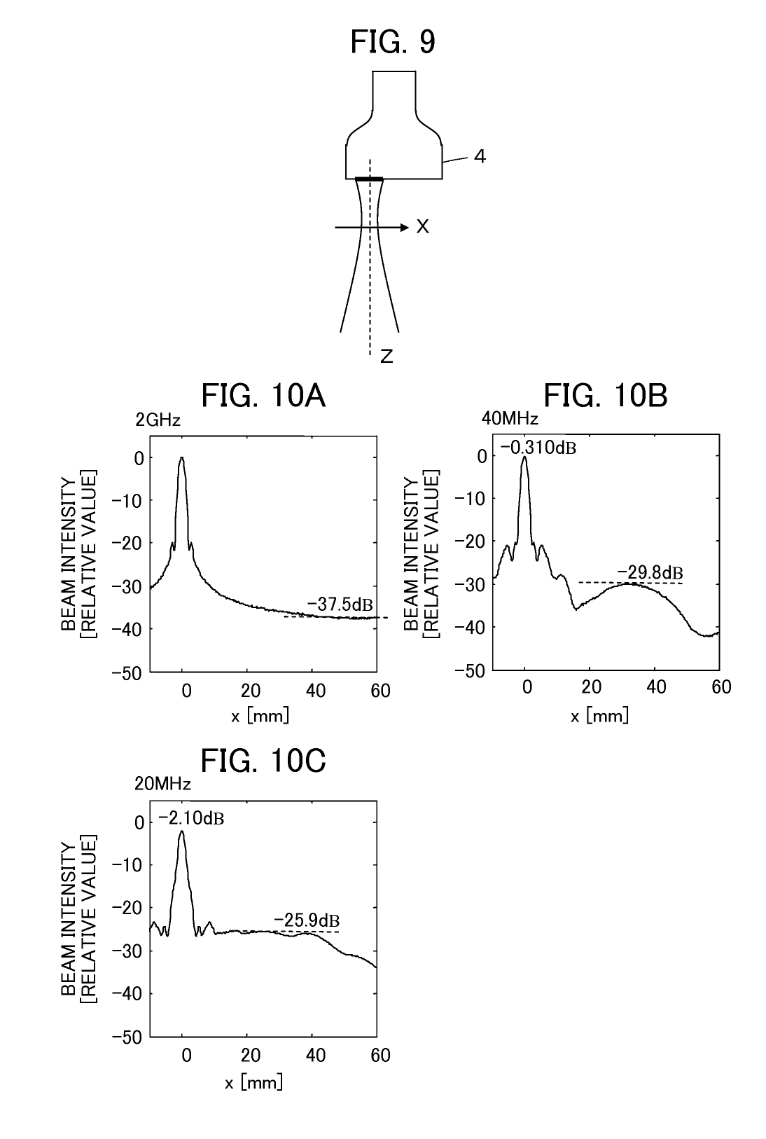

FIGS. 10A to 10C illustrate profiles of synthesized beams in the X direction perpendicular to the direction Z in which the ultrasonic beams travel when the quantization accuracy in delay of the elements of the transducer array is changed as the ultrasonic beams are transmitted from the transducer array of the ultrasound probe 4 as illustrated in FIG. 9. FIGS. 10A, 10B, and 10C illustrate profiles as of the time when the quantization frequency is 2 GHz, 40 MHz, and 20 MHz, respectively. As will be seen from these figures, as the quantization frequency is increased to enhance the quantization accuracy, the peak value increases and the beam floor lowers, enhancing the contrast and thus sharpening the profiles of the synthesized beams, whereas conversely, as the quantization frequency is reduced to lower the quantization accuracy, the profiles of the synthesized beams deteriorate due to quantization error. Therefore, a high accuracy ultrasound image can be obtained by causing the first diagnostic apparatus unit 1 and the second diagnostic apparatus unit 2 to operate in synchronism using the synchronizing clock signal and the main trigger signal.

Although, according to Embodiment 1, the back end 13 of the first diagnostic apparatus unit 1, which is the master apparatus body, produces the image signal, data may be transmitted via the operation control cable 6 from the back end 13 of the first diagnostic apparatus unit 1 to the back end 23 of the second diagnostic apparatus unit 2, so that the back ends 13 and 23 of both diagnostic apparatus units 1 and 2 may cooperate in data processing related to generation of the ultrasound image. Thus, the burden on the back end in the master apparatus body in data processing can be reduced to enable processing at an increased speed.

When the first diagnostic apparatus unit 1 and the second diagnostic apparatus unit 2 each perform a normal ultrasound diagnostic operation independently as in steps S9, S10, and S11 in FIG. 3, the beam former 22 of the second diagnostic apparatus unit 2 is connected to the back end 23 in the second diagnostic apparatus unit 2 as indicated by a dotted line in FIG. 2 in stead of the beam former 22 of the second diagnostic apparatus unit 2 being connected to the back end 13 of the first diagnostic apparatus unit 1 via the data bus 5.

Although the two diagnostic apparatus units 1 and 2 operate in synchronism according to Embodiment 1, the invention is not limited thereto; three or more diagnostic apparatus units may be connected to a single ultrasound probe to achieve synchronized operation thereof wherein one of these diagnostic apparatus units is made to function as master apparatus body while the other remaining diagnostic apparatus units are made to function as slave apparatus bodies. In this case, the synchronizing clock signal and the main trigger signal may be supplied from the diagnostic apparatus unit functioning as master apparatus body to a plurality of diagnostic apparatus units functioning as slave apparatus bodies.

### Embodiment 2

Although, according to Embodiment 1, the first diagnostic apparatus unit 1 and the second diagnostic apparatus unit 2, provided respectively with the back ends 13 and 23 for producing the image signal and the monitors 14 and 24 for displaying the ultrasound image, respectively, perform synchronized operation, the invention is not limited thereto; diagnostic apparatus sub-units not provided with any means for producing the ultrasound image may be used as diagnostic apparatus bodies and connected to a common ultrasound probe to achieve synchronized operation.

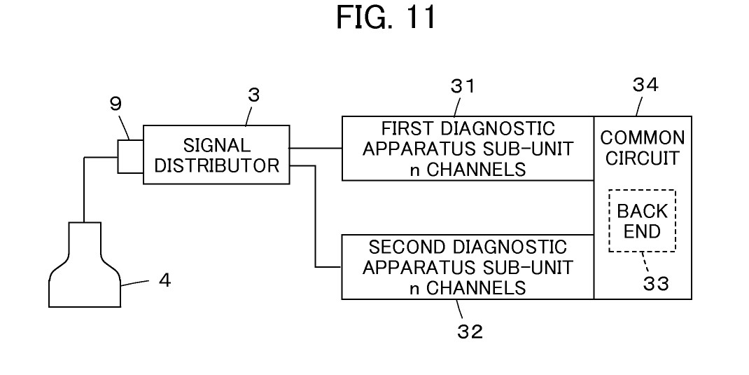

FIG. 11 illustrates a configuration of the ultrasound diagnostic apparatus according to Embodiment 2. This ultrasound diagnostic apparatus comprises a first diagnostic apparatus sub-unit 31 and a second diagnostic apparatus sub-unit 32 as two diagnostic apparatus bodies. These first diagnostic apparatus sub-unit 31 and the second diagnostic apparatus sub-unit 32 are connected via the signal distributor 3 to a common ultrasound probe 4.

The first diagnostic apparatus sub-unit 31 and the second diagnostic apparatus sub-unit 32 have an identical internal configuration to each other and each comprise an n number of channels of ultrasound transmission/reception circuits but are not provided with a back end for producing the ultrasound image as are the first diagnostic apparatus unit 1 with the back end 13 and the second diagnostic apparatus unit 2 with the back end 23 in Embodiment 1. Therefore, the first diagnostic apparatus sub-unit 31 and the second diagnostic apparatus sub-unit 32 are connected to a common circuit 34 provided with a back end 33.

Besides the back end 33, the common circuit 34 comprises a clock retrigger circuit for supplying the synchronizing clock signal and the main trigger signal to both diagnostic apparatus sub-units 31 and 32, as well as a monitor for displaying the ultrasound image.

The first diagnostic apparatus sub-unit 31 and the second diagnostic apparatus sub-unit 32 operate in synchronism according to the synchronizing clock signal and the main trigger signal supplied from the clock retrigger circuit of the common circuit 34 and each produce sample data according to the reception signals outputted from the corresponding transducers of the ultrasound probe 4 to generate the sound ray signals. The sound ray signal generated by the first diagnostic apparatus sub-unit 31 and the sound ray signal generated by the second diagnostic apparatus sub-unit 32 are combined, ana, based on the synthesized sound ray signal, the image signal is produced by the back end 33 of the common circuit 34, whereupon the monitor of the common circuit 34 displays the ultrasound image.

Also with such configuration, the number of reception signals that can be processed simultaneously in parallel with both the diagnostic apparatus sub-units 31 and 32 operating in synchronism is also “2 n” as in Embodiment 1, enabling a high quality ultrasound image to be obtained.

Although two diagnostic apparatus sub-units 31 and 32 are connected to the common ultrasound probe 4 in the configuration shown in FIG. 11, three or more diagnostic apparatus sub-units may be connected to a single ultrasound probe to perform synchronized operation.

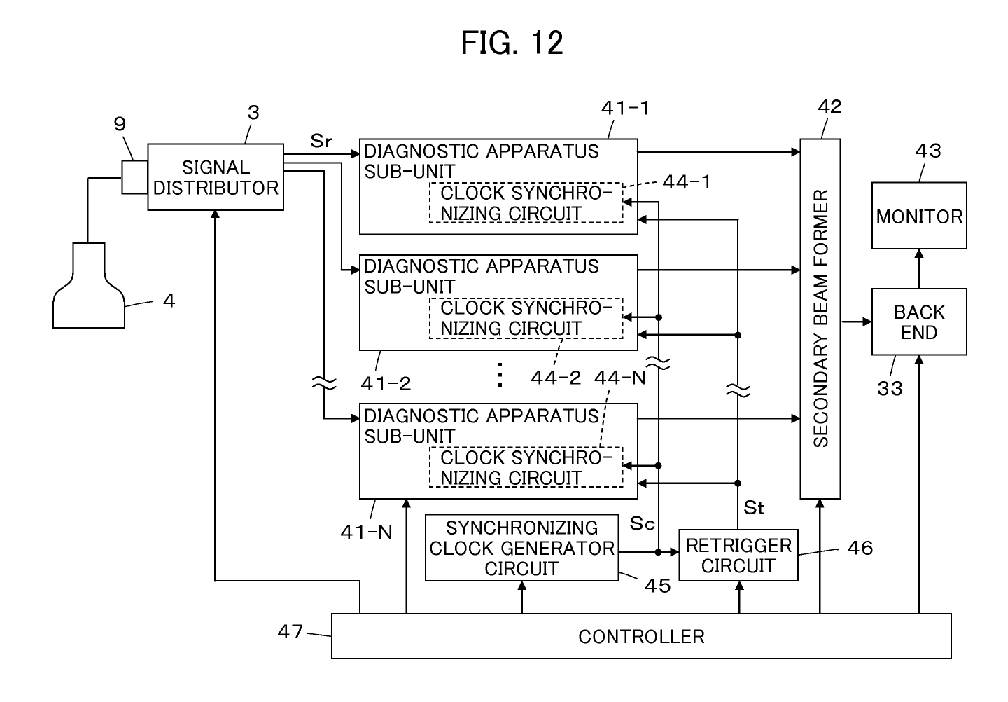

FIG. 12 illustrates a specific configuration of ultrasound diagnostic apparatus wherein an N number of diagnostic apparatus sub-units 41-1 to 41-N are made to perform synchronized operation.

The ultrasound probe 4 is connected via the signal distributor 3 to an N number of diagnostic apparatus sub-units 41-1 to 41-N, which in turn are connected via a secondary beam former 42 to the back end 33, which in turn is connected to a monitor 43. The diagnostic apparatus sub-units 41-1 to 41-N respectively comprise built-in clock synchronizing circuits 44-1 to 44-N, which are connected to a synchronizing clock generator circuit 45, which is connected to a retrigger circuit 46, which in turn is connected to the diagnostic apparatus sub-units 41-1 to 41-N. The signal distributor 3, the diagnostic apparatus sub-units 41-1 to 41-N, the secondary beam former 42, the back end 33, the synchronizing clock generator circuit 45, and the trigger circuit 46 are connected to a controller 47.

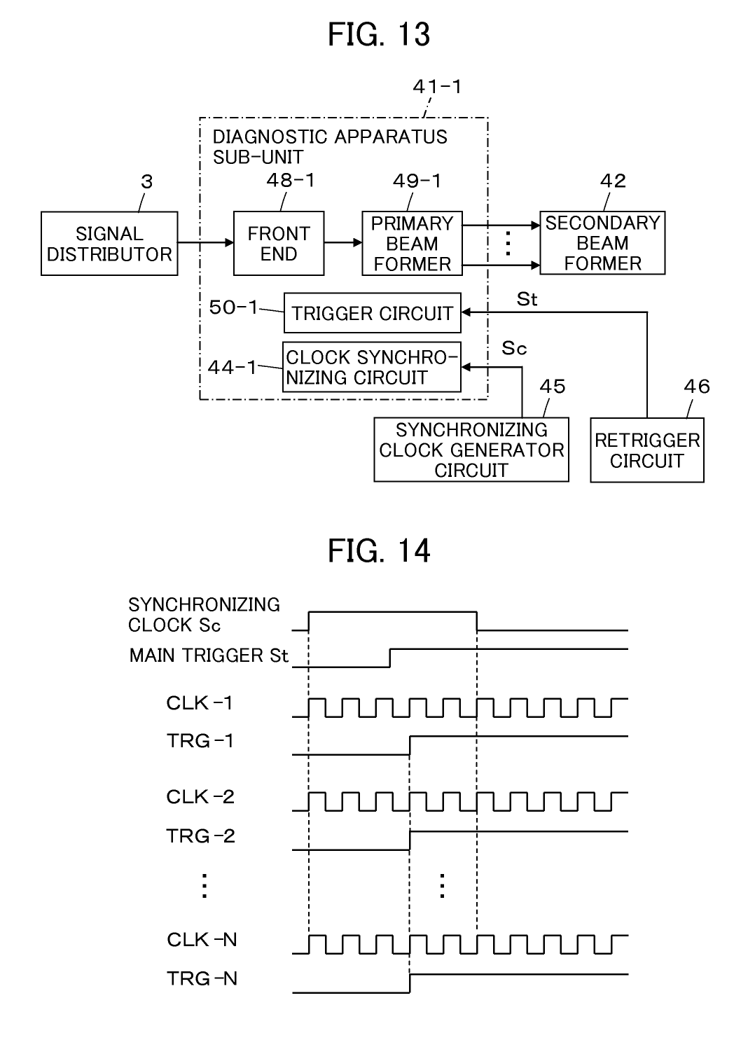

As illustrated in FIG. 13, the diagnostic apparatus sub-unit 41-1 comprises, besides the clock synchronizing circuit 44-1, a front end 48-1 connected to the signal distributor 3 and a primary beam former 49-1 connected to the front end 48-1; the primary beam former 49-1 is connected to the secondary beam former 42. The diagnostic apparatus sub-unit 41-1 further comprises a trigger circuit 50-1 connected to the retrigger circuit 46.

Like the front ends 11 and 21 in Embodiment 1, the front end 48-1 supplies actuation signals to the corresponding transducers of the ultrasound probe 4, which is connected to the front end 48-1 via the signal distributor 3, receives ultrasonic echoes returning from a subject to perform quadrature detection or other processing on reception signals generated by these transducers to produce a complex baseband signal, and performs sampling on the complex baseband signal to produce sample data containing information on an area of a tissue. The front end 48-1 may perform data compression processing for high efficiency encoding on the data obtained by sampling the complex baseband signal.

Like the beam formers 12 and 22 in Embodiment 1, the primary beam former 49-1 selects one reception delay pattern from a plurality of previously stored reception delay patterns according to the reception direction set by the controller 47 and, based on a selected reception delay pattern, performs the reception focusing processing by performing addition by providing respective delays in the plurality of complex baseband signals represented by the sample data, and produces and supplies a sound ray signal to the secondary beam former 42.

Like the diagnostic apparatus sub-unit 41-1 illustrated in FIG. 13, the other diagnostic apparatus sub-units 41-2 to 41-N respectively comprise front ends, primary beam formers, and trigger circuits in addition to clock synchronizing circuits 44-2 to 44-N.

The secondary beam former 42 produces a synthesized sound ray signal obtained by combining the sound ray signals produced by the respective primary beam formers of the diagnostic apparatus sub-units 41-1 to 41-N.

The back end 33 produces a B-mode image signal, which is tomographic image information on the tissue of the subject, according to the synthesized sound ray signal generated by the secondary beam former 42.

The monitor 43 displays an ultrasound diagnostic image based on an image signal produced by the back end 33.

The synchronizing clock generator circuit 45 generates a common synchronizing clock signal Sc for causing the diagnostic apparatus sub-units 41-1 to 41-N to operate in synchronism and supplies the signal Sc to the diagnostic apparatus sub-units 41-1 to 41-N. Preferably, the synchronizing clock signal Sc has a frequency that is at least double the major central frequency used by the ultrasound probe 4 so that its frequency does not coincide with the frequency band of the ultrasound probe 4.

As illustrated in FIG. 14, the clock synchronizing circuits 44-1 to 44-N built in the diagnostic apparatus sub-units 41-1 to 41-N generate high-frequency clock signals CLK-1 to CLK -N in synchronism with each other and necessary to operate the A/D converters (analog-to-digital converters) built in the front ends according to the synchronizing clock signal Sc generated by the synchronizing clock generator circuit 45.

The retrigger circuit 46 supplies the diagnostic apparatus sub-units 41-1 to 41-N with a main trigger signal St triggered by the synchronizing clock signal Sc generated by the synchronizing clock generator circuit 45. As illustrated in FIG. 14, the trigger circuits each built in the diagnostic apparatus sub-units 41-1 to 41-N generate trigger signals TRG-1 to TRG-N that are in synchronism with each other based on the main trigger signal St supplied from the retrigger circuit 46 and the clock signals CLK-1 to CLK-N produced by the clock synchronizing circuits 44-1 to 44-N.

Further, the controller 47 controls operations of components provided inside the ultrasound diagnostic apparatus.

Next, the operation of the ultrasound diagnostic apparatus illustrated in FIG. 12 will be described.

The diagnostic apparatus sub-units 41-1 to 41-N operate in synchronism according to the clock signals produced by the clock synchronizing circuits CLK-1 to CLK-N and the trigger signals TRG-1 to TRG-N, supply actuation signals from the respective front ends to the corresponding transducers of the ultrasound probe 4 to cause ultrasonic waves to be transmitted, produce sample data according to reception signal Sr outputted from the transducers having received ultrasonic echoes from the subject, and generate sound ray signals in the primary beam formers. The sound ray signals generated by the respective primary beam formers of the diagnostic apparatus sub-units 41-1 to 41-N are combined by the secondary beam former 42 to produce the synthesized sound ray signal and, based on the synthesized sound ray signal, the image signal is produced by the back end 33, whereupon the monitor 43 displays the ultrasound diagnostic image.

The synchronized operation of an N number of the diagnostic apparatus sub-units 41-1 to 41-N increases the number of reception signals that can be processed simultaneously in parallel and, as in Embodiment 1, enables a high quality ultrasound image to be obtained.

Also in this Embodiment 2 as in Embodiment 1, the diagnostic apparatus sub-units 41-1 to 41-N may be connected to the signal distributor 3 via the respective unit side connectors, and, according to the ID numbers assigned to the unit side connectors, one of the diagnostic apparatus sub-units 41-1 to 41-N may be caused to function as master apparatus body while the other remaining diagnostic apparatus sub-units may be caused to function as slave apparatus bodies to achieve synchronized operation of the diagnostic apparatus sub-units 41-1 to 41-N.

### Embodiment 3

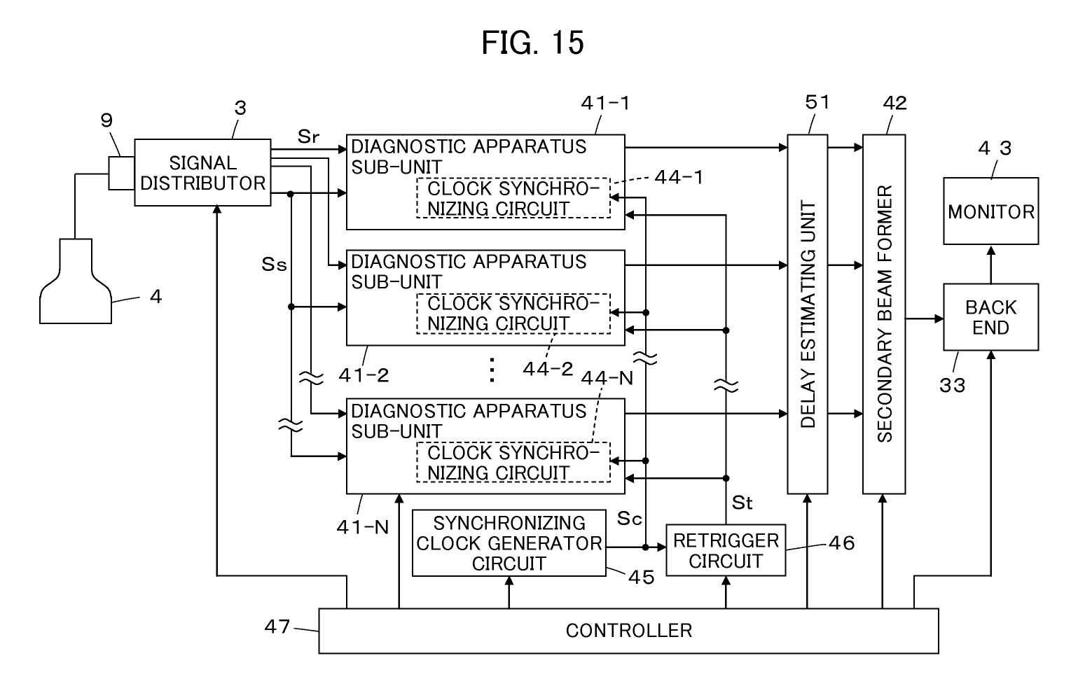

FIG. 15 illustrates a specific configuration of the ultrasound diagnostic apparatus according to Embodiment 3. As compared with the apparatus according to Embodiment 2 illustrated in FIG. 12, the ultrasound diagnostic apparatus shown in FIG. 15 additionally comprises a delay estimating unit 51 connected between the diagnostic apparatus sub-units 41-1 to 41-N and the secondary beam former 42 and further differs in that the reception signal from one transducer of the ultrasound probe 4 is inputted via the signal distributor 3 as identical signal Ss to the diagnostic apparatus sub-units 41-1 to 41-N under the control of the controller 47.

The delay estimating unit 51 estimates the clock skew occurring among the diagnostic apparatus sub-units 41-1 to 41-N based on the processing results yielded by the diagnostic apparatus sub-units 41-1 to 41-N when the identical signal Ss is inputted to the diagnostic apparatus sub-units 41-1 to 41-N, i.e., based on the sound ray signals produced respectively by the primary beam formers of the diagnostic apparatus sub-units 41-1 to 41-N. The estimation of this clock skew is performed after one round of transmission and reception of ultrasonic waves from the ultrasound probe 4 has been completed.

The secondary beam former 42 combines the sound ray signals to produce a synthesized sound ray signal by making correction so as to minimize the effects of the clock skew based on the clock skew estimated by the delay estimating unit 51.

Thus estimating the clock skew occurring among the diagnostic apparatus sub-units 41-1 to 41-N and producing a synthesized sound ray signal based on the clock skew enable an ultrasound image with a still higher accuracy to be obtained.

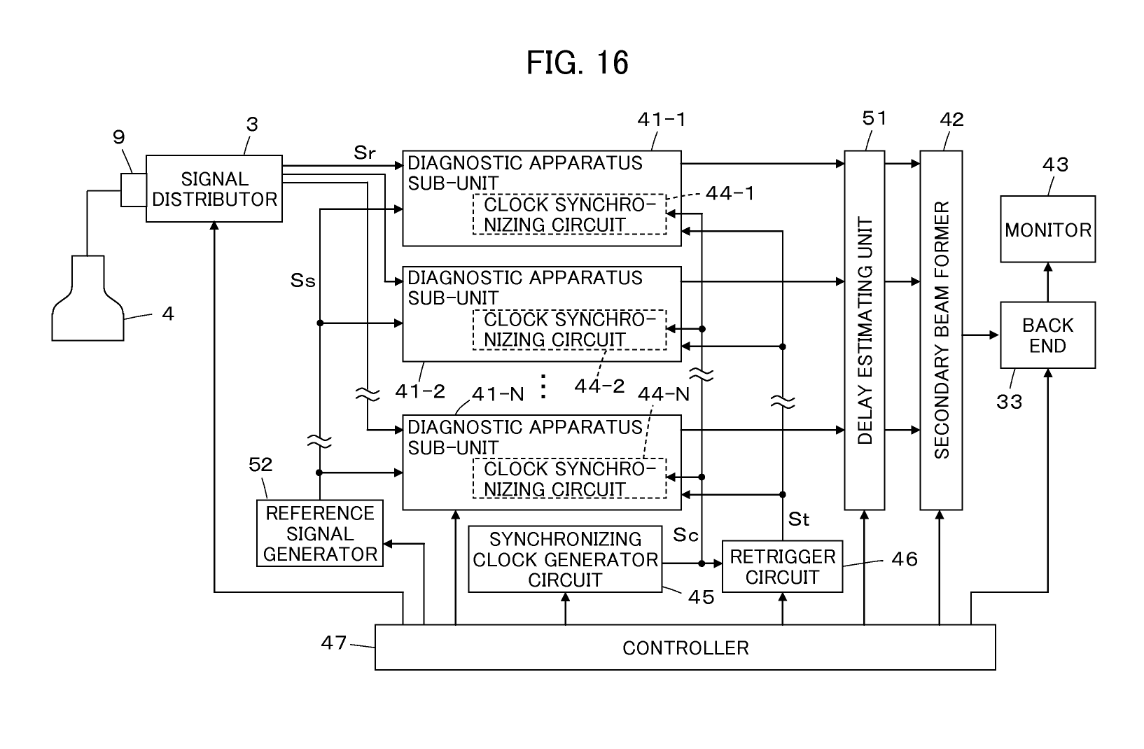

Although the reception signal from one transducer of the ultrasound probe 4 is inputted to the diagnostic apparatus sub-units 41-1 to 41-N as identical signal Ss in the ultrasound diagnostic apparatus illustrated in FIG. 15, a reference signal generator 52 may be additionally provided as illustrated in FIG. 16, so that the reference signal generator 52 may input the identical signal Ss to the diagnostic apparatus sub-units 41-1 to 41-N.

The reference signal generator 52 produces a reference signal, which is inputted to the diagnostic apparatus sub-units 41-1 to 41-N as identical signal Ss.

Also with such configuration, the delay estimating unit 51 can estimate the clock skew occurring among the diagnostic apparatus sub-units 41-1 to 41-N when the identical signal Ss is inputted to the diagnostic apparatus sub-units 41-1 to 41-N for the secondary beam former 42 to produce a synthesized sound ray signal based on the clock skew estimated by the delay estimating unit 51.

The reference signal generator 52 may be so adapted to input the reference signal it produces to the diagnostic apparatus sub-units 41-1 to 41-N as identical signal Ss at all times, so that the delay estimating unit 51 may estimate the clock skew after one round of transmission and reception of ultrasonic waves from the ultrasound probe 4 has been completed. Alternatively, the reference signal generator 52 may be so adapted to input the reference signal to the diagnostic apparatus sub-units 41-1 to 41-N as identical signal Ss only at a given time preceding the transmission of the ultrasonic waves from the transducer array of the ultrasound probe 4, so that the delay estimating unit 51 may estimate the clock skew at a timing corresponding to said given time.

Although the retrigger circuit 46 supplies the main trigger signal St triggered by the synchronizing clock signal Sc generated by the synchronizing clock generator circuit 45 to the diagnostic apparatus sub-units 41-1 to 41-N in the ultrasound diagnostic apparatus in above Embodiments 2 and 3, a trigger circuit that is not connected to the synchronizing clock generator circuit 45 may be connected, in stead of the trigger circuit 46, to the diagnostic apparatus sub-units 41-1 to 41-N so that this trigger circuit may supply the main trigger signal St to the diagnostic apparatus sub-units 41-1 to 41-N.

However, it is preferable to supply the main trigger signal St triggered by the synchronizing clock signal Sc in the retrigger circuit 46 to the diagnostic apparatus sub-units 41-1 to 41-N as in Embodiments 2 and 3 because the synchronism in operation among the diagnostic apparatus sub-units 41-1 to 41-N is then enhanced.

What is claimed is:

1. An ultrasound diagnostic apparatus comprising:
an ultrasound probe including a transducer array;
a plurality of diagnostic apparatus bodies corresponding to a plurality of parts of the transducer array for transmitting ultrasonic waves through corresponding transducers and processing reception signals from the corresponding transducers, respectively; and
a synchronizing signal supply means for supplying a common clock signal and a common trigger signal to the plurality of diagnostic apparatus bodies for causing the plurality of diagnostic apparatus bodies to operate in synchronism.

2. The ultrasound diagnostic apparatus according to claim 1, further comprising a back end for producing an ultrasound image based on reception signals respectively processed by the plurality of diagnostic apparatus bodies.

3. The ultrasound diagnostic apparatus according to claim 2, wherein the synchronizing clock supply means comprises a synchronizing clock generator circuit for producing the common clock signal and a trigger circuit for producing the common trigger signal.

4. The ultrasound diagnostic apparatus according to claim 3, wherein the trigger circuit produces the common trigger signal based on the common clock signal produced by the synchronizing clock generator circuit.

5. The ultrasound diagnostic apparatus according to claim 1, wherein the plurality of diagnostic apparatus bodies each have incorporated therein a back end for producing an ultrasound image based on reception signals transmitted from the corresponding transducers.

6. The ultrasound diagnostic apparatus according to claim 5,
wherein the plurality of diagnostic apparatus bodies each have a clock circuit for producing a clock signal and a trigger circuit for producing a trigger signal, and
the synchronizing signal supply means comprises the clock circuit and the trigger circuit incorporated in one diagnostic apparatus body selected as master apparatus body from among the plurality of diagnostic apparatus bodies.

7. The ultrasound diagnostic apparatus according to claim 6,
wherein the other diagnostic apparatus bodies than the master apparatus body among the plurality of diagnostic apparatus bodies respectively transmit results obtained by processing reception signals transmitted from corresponding transducers to the master apparatus body, and
the back end incorporated in the master apparatus body produces an ultrasound image based on results obtained by processing reception signals produced by all the diagnostic apparatus bodies.

8. The ultrasound diagnostic apparatus according to claim 7, wherein the plurality of diagnostic apparatus bodies cooperate in data processing related to generation of the ultrasound image.

9. The ultrasound diagnostic apparatus according to claim 1, wherein the common clock signal has a frequency at least twice as high as a major central frequency used by the ultrasound probe.

10. The ultrasound diagnostic apparatus according to claim 1, further comprising a delay estimating unit for estimating a clock skew occurring among the plurality of diagnostic apparatus bodies based on results obtained by processing performed by the plurality of diagnostic apparatus bodies for an identical signal entered in the plurality of diagnostic apparatus bodies.

11. The ultrasound diagnostic apparatus according to claim 10, wherein the identical signal is a reception signal from an identical transducer of the transducer array.

12. The ultrasound diagnostic apparatus according to claim 10, further comprising a reference signal generator for producing a reference signal and entering the reference signal in the plurality of diagnostic apparatus bodies as the identical signal.

13. The ultrasound diagnostic apparatus according to claim 12, wherein the reference signal generator enters the reference signal in the plurality of diagnostic apparatus bodies at all times.

14. The ultrasound diagnostic apparatus according to claim 12, wherein the reference signal generator enters the reference signal in the plurality of diagnostic apparatus bodies only at a given time preceding the transmission of ultrasonic waves from the transducer array.

15. An ultrasound diagnostic apparatus comprising:
an ultrasound probe including a transducer array; and
a plurality of diagnostic apparatus bodies corresponding to a plurality of parts of the transducer array for transmitting ultrasonic waves through corresponding transducers and processing reception signals from the corresponding transducers, respectively,
wherein when the one ultrasound probe is connected to the plurality of diagnostic apparatus bodies, one diagnostic apparatus body is selected as master apparatus body from among the plurality of diagnostic apparatus bodies while the other diagnostic apparatus bodies become slave apparatus bodies for the master apparatus body, and the plurality of diagnostic apparatus bodies operate in synchronism.

16. The ultrasound diagnostic apparatus according to claim 15, wherein the plurality of diagnostic apparatus bodies each have incorporated therein a back end for producing an ultrasound image based on reception signals transmitted from the corresponding transducers.

17. The ultrasound diagnostic apparatus according to claim 16, wherein the master apparatus body supplies a common clock signal and a common trigger signal to the slave apparatus bodies.

18. The ultrasound diagnostic apparatus according to claim 17,
wherein the plurality of diagnostic apparatus bodies each have a clock circuit for producing a clock signal and a trigger circuit for producing a trigger signal, and
the master apparatus body supplies the slave apparatus bodies with a clock signal produced by the incorporated clock circuit as the common clock signal and a trigger signal produced by the incorporated trigger circuit based on the common clock signal as the common trigger signal.

19. The ultrasound diagnostic apparatus according to claim 16,
wherein the slave apparatus bodies transmit results obtained by processing reception signals transmitted from corresponding transducers to the master apparatus body, and
the back end incorporated in the master apparatus body produces an ultrasound image based on results obtained by processing reception signals produced by all the diagnostic apparatus bodies.

20. The ultrasound diagnostic apparatus according to claim 19, wherein the plurality of diagnostic apparatus bodies cooperate in data processing related to generation of the ultrasound image.

21. The ultrasound diagnostic apparatus according to claim 15, further comprising a back end for producing an ultrasound image based on reception signals respectively processed by the plurality of diagnostic apparatus bodies.

22. The ultrasound diagnostic apparatus according to claim 21, further comprising:
a synchronizing clock generator circuit for producing a common clock signal for causing the plurality of diagnostic apparatus bodies to operate in synchronism and supplying the common clock signal to the plurality of diagnostic apparatus bodies; and
a trigger circuit for producing a common trigger signal based on a common clock signal produced by the synchronizing clock generator circuit and supplying the common trigger signal to the plurality of diagnostic apparatus bodies.
